(19) United States
(12) Patent Application Publication
(10) Pub. No.: US 2018/0070835 A1
(43) Pub. Date: Mar. 15, 2018

(54) TUBE FOR BLOOD PRESSURE CUFF AND BLOOD PRESSURE CUFF

(71) Applicant: Nihon Kohden Corporation, Tokyo (JP)

(72) Inventors: Teishi Oishi, Tokyo (JP); Takashi USUDA, Tokyo (JP)

(21) Appl. No.: 15/597,413

(22) Filed: May 17, 2017

(30) Foreign Application Priority Data
Sep. 9, 2016 (JP) 2016-177017

(51) Int. Cl. A61B 5/0235; A61B 5/00

(52) U.S. Cl. CPC A61B 5/0235; A61B 5/742

(57) ABSTRACT

A tube for a blood pressure cuff includes a hollow and long shape, and plural projection portions projecting toward a center of a regular polygon in correspondence with vertices of the regular polygon which inscribes in an outer circumference and has 5 or more angles, in a shape of an inner circumference in a cross section perpendicular to a longitudinal direction of the cuff tube.

### CROSS-REFERENCE TO RELATED APPLICATION

This application claims a priority under the Paris Convention of Japanese patent application No. 2016-177017 filed on Sep. 9, 2016, the entirety of which is incorporated herein by references.

### BACKGROUND

The presently disclosed subject matter relates to a tube for a blood pressure cuff and a blood pressure cuff.

In a blood pressure measurement by using the oscillometric method, a cuff pressure applied to an upper arm of a patient by a cuff wrapped to the upper arm is set to a value higher than a systolic blood pressure, and the upper arm is pressurized to a target value by sending air into the cuff from a sphygmomanometer body through a tube. Then, a blood pressure is measured by gradually discharging the air from the cuff through the tube to reduce the cuff pressure and detecting an oscillation generated in the cuff due to a pulse wave of the patient.

However, the tube connecting the cuff and the sphygmomanometer may be bent and kinked as a result of, for example, the patient changing the body position thereof during blood pressure measurement, and an air flow path the tube may thus be occluded. If the air flow path in the tube is occluded, air cannot be sent into the cuff, and the blood pressure measurement cannot be performed. Further, air cannot be discharged from the cuff, and thus the applied cuff pressure is kept without being reduced. This may continuously put a relatively large strain on the upper arm of the patient.

Unexamined Japanese Patent Publication No. 2004-223102 discloses a cuff tube for preventing the occlusion of the tube. That is, an elastic hose constituting the tube is configured so as to have an inner circumferential shape that is an approximate triangle or an approximate pentagon constituted by plural straight line portions and plural arc portions interconnecting the plural straight line portions in a cross section. In addition, in the cross section, the thickness of the tube corresponding to the interval between the inner circumference and the outer circumference of a circular shape, is configured to be contrary between symmetrical positions with respect to the central axis of the tube. According to this, the tube is less likely to be bent in the blood pressure measurement, and an air flow path in the cross section is secured even in the case where the tube is bent.

### SUMMARY

However, in the case where the inner circumferential shape of the tube in the cross section is configured to be a shape different from a circle as in the related art described above, the air flow path in the tube may still be occluded depending on a direction in which the tube is bent. In other words, in the case of the tube having an approximately triangular inner circumferential shape in the cross section, possibility of the tube being occluded may be different between a case where the tube is bent along a straight line passing through an arc portion and a straight line portion which is opposed to the arc portion across the center axis and a case where the tube is bent along a straight line passing through a straight line portion and another straight line portion which is opposed to the straight line portion across the center axis. Therefore, the tube may be occluded when bent in a certain direction even if the tube is not occluded when bent in another direction.

Therefore, the presently disclosed subject matter is to provide a tube for a blood pressure cuff that is capable of reducing inclination of occlusion due to the bending direction such as the difference in occurrence of occlusion depending on the bending direction of the tube, and capable of preventing the occlusion of the tube regardless of the bending direction.

An aspect of the tube for a blood pressure cuff reflecting one aspect of the presently disclosed subject matter comprises a hollow and long shape, and plural projection portions projecting toward a center of a regular polygon in correspondence with vertices of said regular polygon which inscribes in an outer circumference and has 5 or more angles, in a shape of an inner circumference in a cross section perpendicular to a longitudinal direction of the cuff tube.

### BRIEF DESCRIPTION OF THE DRAWINGS

FIG. 1 is a block diagram of a blood pressure measurement apparatus using a blood pressure cuff and a tube for a blood pressure cuff according to an embodiment of the presently disclosed subject matter;

FIG. 2 is a perspective view of the cuff illustrating a configuration thereof;

FIG. 3 includes a cross section view of the tube according to the embodiment of the presently disclosed subject matter of cutting along the longitudinal direction of the tube and side views of both ends of the tube;

FIG. 4 is a cross section view of the tube of cutting along A-A′ illustrated in FIG. 3;

FIG. 5 is a cross section view of a deformed section portion of the tube according to another exemplary embodiment of the presently disclosed subject matter;

FIG. 6 illustrates a state in which the tube is bent; and

FIG. 7 illustrates a configuration of the tube in which a relay portion is provided on an end of a straight portion of the tube.

### DETAILED DESCRIPTION

A tube for a blood pressure cuff and a blood pressure cuff according to an embodiment of the presently disclosed subject matter will be described below with reference to drawings. The dimensional ratio in the drawings is exaggerated for the convenience of description, and may be different from the actual ratio.

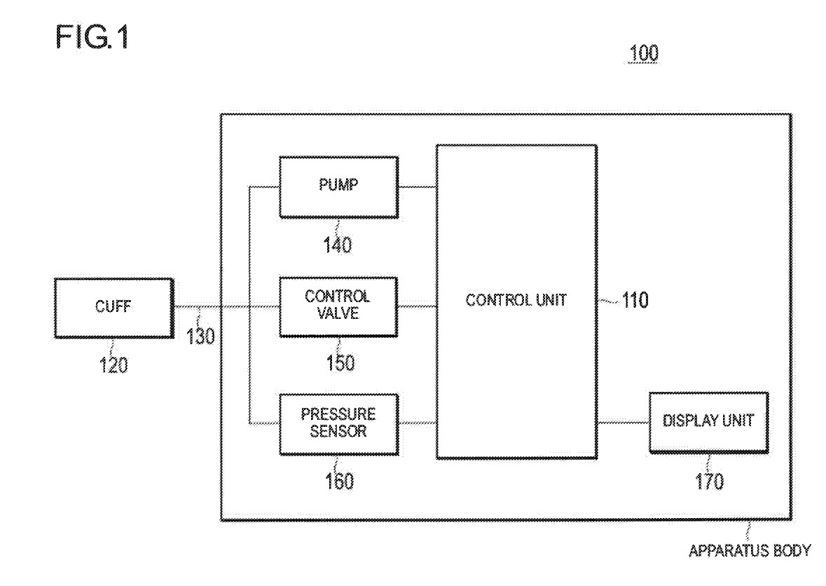

FIG. 1 is a block diagram of a blood pressure measurement apparatus using the blood pressure cuff and the tube for the blood pressure cuff according to the embodiment of the presently disclosed subject matter. A blood pressure measurement apparatus 100 performs measurement of blood pressure by using, for example, an oscillometric method.

The blood pressure measurement apparatus 100 can include a control unit 110, a cuff 120, a tube 130, a pump 140, a control valve 150, a pressure sensor 160, and a display unit 170. The tube 130 constitutes the tube for the blood pressure cuff. The cuff 120 and the tube 130 constitute the blood pressure cuff.

The control unit 110, the pump 140, the control valve 150, the pressure sensor 160, and the display unit 170 may be included in an apparatus body of the blood pressure measurement apparatus 100. The tube 130 is connected to a connector of the apparatus body, and the apparatus body is thereby connected to the cuff 120 via the tube 130.

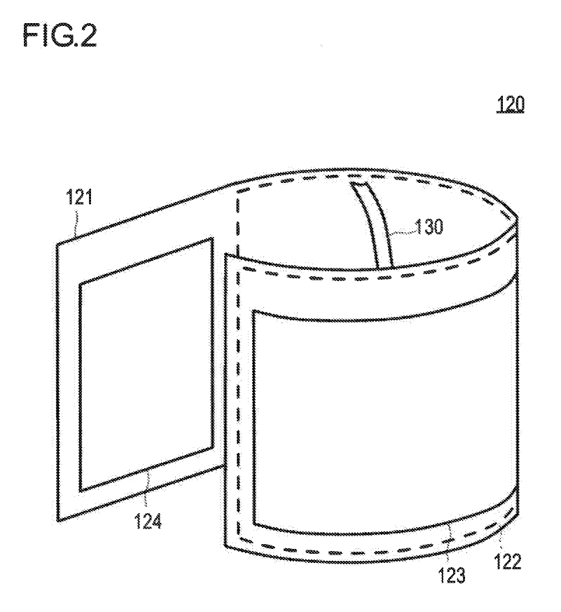

FIG. 2 is a perspective view of the cuff illustrating a configuration thereof.

The cuff 120 can include a body 121, a bladder 122, the tube 130, and a hook and loop fastener having a hook surface 123 and a loop surface 124.

The cuff 120 can be wrapped around an upper arm of a patient in blood pressure measurement by binding the hook surface 123 and the loop surface 124 together. For simplicity of description, the description will be given hereinbelow on the premise that the cuff 120 is wrapped to the upper arm of the patient.

The body 121 of the cuff 120 may be formed of a synthetic resin such as polyester or nylon.

The bladder 122 may be formed by forming a part of the body 121 to have a double-layered structure and fusing the periphery to provide a sealed inner space.

The tube 130 has a hollow and long shape and is formed of an elastic material. The tube 130 may be formed of, for example, polyvinyl chloride, polyethylene, or polypropylene. The tube 130 includes, in the shape of the inner circumference in a cross section perpendicular to the longitudinal direction (hereinafter referred to as a “cross section”), plural projection portions projecting toward the center of a regular polygon in correspondence with respective vertices of the regular polygon. The regular polygon includes 5 or more angles and is inscribed in the outer circumference of the tube in the cross section. To be noted, the regular polygon does not have to be inscribed in the outer circumference.

The tube 130 is connected to the bladder 122 such that the bladder 122 communicates with an air flow path 134 of the tube 130. A flange 132 integrally formed in one piece with the tube 130 may be provided on the periphery of one end of the tube 130. The flange 132 may have, for example, a collar shape in which the flange 132 projects around the tube 130 like eaves. The flange 132 on the end of the tube 130 is inserted in the inner space of the bladder 122 through a small opening portion provided in the bladder 122, and the flange 132 is fused with the periphery of the small opening portion. As a result of this, the air flow path 134 of the tube 130 is communicably connected with the inner space of the bladder 122, so that the tube 130 and the bladder 122 are connected to each other.

The flange 132 may not be integrally formed in one piece with the tube 130. For example, separately from the tube 130, a relatively short tube-like component formed of nylon (registered trademark) in the periphery of one end of which a flange is provided may be produced, and the flange may be inserted in the small opening portion of the bladder 122 and fused with the periphery of the small opening portion. Then, the tube 130 and the bladder 122 may be connected by inserting the other end of the tube-like component in the lumen of the tube 130 and fusing the other end with the tube 130.

Referring back to FIG. 1, the pump 140 pressurizes the cuff 120 wrapped to the upper arm of the patient by sending air into the bladder 122 of the cuff 120 wrapped to the upper arm of the patient through the tube 130.

The air bladder is inflated or deflated by the control valve 150 which is opened and closed.

The pressure sensor 160 detects the pressure of the air inside the bladder 122 of the cuff 120 (hereinafter referred to as “cuff internal pressure”), and transmits the result of detection to the control unit 110. The pressure sensor 160 is, for example, a piezoresistive pressure sensor, and transmits the detected cuff internal pressure to the control portion 110 as an electric signal.

The control unit 110 may be constituted by a central processing unit (CPU), a program, and a random access memory (RAM). The control portion 110 executes various arithmetic processing by the CPU in accordance with the program by using the RAM as a work area, and controls each constituent of the blood pressure measurement apparatus 100.

When the pump 140 pressurizes the cuff 120, the control unit 110 calculates a cuff pressure applied to the upper arm of the patient based on the cuff internal pressure detected by the pressure sensor 160. The control unit 110 controls the flow rate of the air sent into the cuff 120 from the pump 140 based on the calculated cuff pressure. The control unit 110 raises the pressure of the cuff 120 until the cuff pressure reaches a pressurizing target value set to a higher value than a systolic blood pressure of the patient.

When reducing the cuff pressure by controlling the control valve 150, the control unit 110 calculates a pressure reduction range of the cuff pressure applied to the upper arm of the patient based on the cuff internal pressure detected by the pressure sensor 160. The control unit 110 controls the flow rate of the air discharged from the cuff 120 by controlling the control valve 150 based on the calculated pressure reduction range of the cuff 120. In addition, the control unit 110 calculates the amplitude of a pulse wave of the patient based on the cuff internal pressure detected by the pressure sensor 160, and further calculates the systolic blood pressure and a diastolic blood pressure based on a change in the amplitude of the pulse wave.

The display unit 170 is constituted by, for example, a liquid crystal display, and displays the calculated systolic blood pressure and diastolic blood pressure.

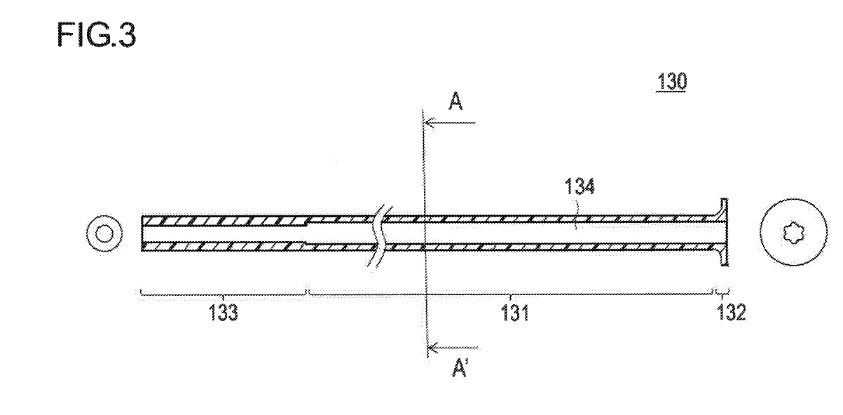

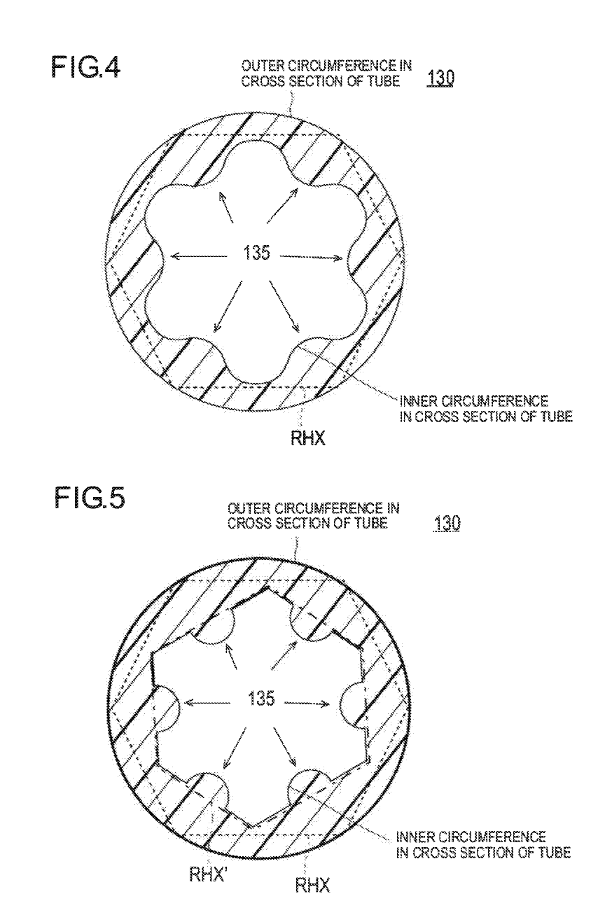

FIG. 3 includes a cross section view of the tube 130 according to the embodiment of the presently disclosed subject matter of cutting along the longitudinal direction of the tube 130 and side views of both ends of the tube 130. FIG. 4 is a cross section view of the tube 130 of cutting along A-A′ illustrated in FIG. 3.

The tube 130 includes a deformed section portion 131 which is a region including plural projection portions 135 on the inner circumference thereof in the cross section of cutting along A-A′. In addition, the tube 130 may include a straight portion 133 (circular section) which does not have the projection portions 135 in the shape of the inner circumference in the cross section in a region within a predetermined distance from one end thereof in the longitudinal direction. According to this, it is not required that a connector of the apparatus body is provided with a recess corresponding to the projection portions 135 and the tube 130 is connected to the connector by inserting the tube 130 in the connector by matching the projection portions 135 with the recess. Thus, complication of the connection process can be prevented, and connectivity as good as a normal tube can be maintained. A distance sufficient for connecting the tube 130 to the connector of the apparatus body may be set as the predetermined distance.

The deformed section portion 131 includes, in the shape of the inner circumference of the tube 130 in the cross section, the plural projection portions 135 projecting toward the center of a regular polygon RHX in correspondence with respective vertices of the regular polygon RHX. The regular polygon RHX includes 5 or more angles and is inscribed in the circular outer circumference. In other words, the deformed section portion 131 has a structure in which the wall thickness periodically increases at every predetermined angle (60 degrees in the case where the regular polygon RHX is a regular hexagon) relative Lo the center axis of the tube 130 along the inner wall surrounding the air flow path 134.

The plural projection portions 135 in the shape of the inner circumference of the tube 130 in the cross section are preferably provided in correspondence with respective vertices of a regular hexagon inscribed in the outer circumference.

The tube 130 is produced by, for example, injection molding of polyvinyl chloride. Specifically, a cavity of a mold for molding polyvinyl chloride into a long cylindrical shape is filled with polyvinyl chloride by injecting the polyvinyl chloride into the cavity. At this time, a long pin for making the long shape into a hollow shape is disposed at the center of the cavity so as to penetrate the polyvinyl chloride filling the cavity. Then, after undergoing a cooling process, the mold is released, and the pin is pulled out of a molded article. Straight-line recesses for forming the plural projection portions 135 projecting in the cross section of the tube 130 are provided on the surface of the pin. In the case of providing the straight portion 133, the straight-line recesses for forming the plural projection portions 135 projecting in the cross section of the tube 130 in a region within a predetermined distance from one end of the tube 130 in the longitudinal direction, are not provided on the surface of the pin.

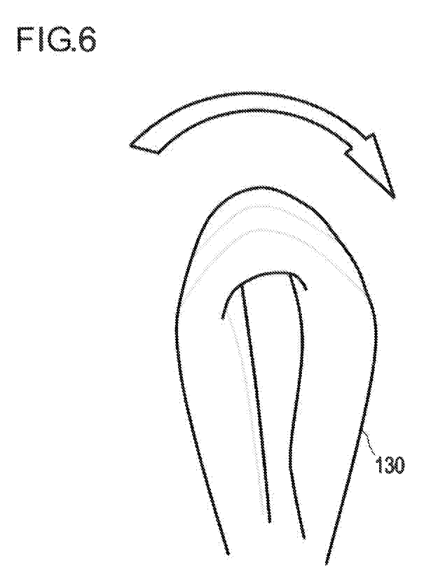

FIG. 5 is a cross section view of the deformed section portion 131 of the tube 130 according to another embodiment of the presently disclosed subject matter.

The tube 130 illustrated in FIG. 5 is similar to the tube 130 illustrated in FIG. 4 in including, in the shape of the inner circumference of the tube 130 in the cross section, the plural projection portions 135 projecting toward the center of a regular polygon RHX in correspondence with respective vertices of the regular polygon RHX. The regular polygon RHX includes 5 or more angles and is inscribed in the outer circumference. However, the tube 130 illustrated in FIG. 5 is different from the tube 130 illustrated in FIG. 4 in that the shape of the inner circumference of the tube 130 in the cross section includes the projection portions 135 in respective sides of another regular polygon RHX′ having the same number of angles as the regular polygon RHX. The shape of the projection portions 135 in the shape of the inner circumference of the tube 130 in the cross section illustrated in FIG. 5 may be a semicircular shape projecting from each side of the other regular polygon RHX′ having the same number of angles as the regular polygon RHX.

FIG. 6 illustrates a state in which the tube 130 is bent.

According to the structure of the deformed section portion 131 described above, the tube 130 is not occluded even when bent by 180 degrees and thus air flows therethrough in a direction indicated by an arrow. This is because an area of flow path to let the air pass through the tube 130 is secured by providing the plural projection portions 135 in the tube 130 in the cross section when the tube 130 is bent.

Further, according to the structure of the deformed section portion 131 described above, the tube 130 is not occluded and thus air flows therethrough regardless of the direction in which the tube 130 is bent. This is because the number of the projection portions 135 is set to 5 or more corresponding to the vertices of the regular polygon RHX having 5 or more angles as a result of the diligence of the present inventors having confirmed the following fact. That is, the inclination of occlusion according to the bending direction depends on the number of the projection portions 135 of the inner circumference of the tube 130 in the cross section provided in correspondence with the vertices of the regular polygon RHX inscribed in the outer circumference in the cross section. The inclination of occlusion does not occur by setting the number of the projection portions 135 to 5 or more in correspondence with the vertices of the regular polygon RHX having 5 or more angles. In contrast, in the case where the projection portions 135 are provided in correspondence with vertices of a regular polygon RHX having less than 5 angles, the inclination of occlusion occurs.

The cause of this is considered to be as follows. That is, in the case where the projection portions 135 are provided in correspondence with the regular polygon RHX having less than 5 angles, the tube 130 is twisted when the tube 130 is bent because the bending force is escaped to a part that can be easily deformed. The degree of the twisting depends on the bending direction. The tube 130 becomes more likely to be occluded when twisted, thus the tube 130 is occluded when bent in a direction in which the degree of twisting becomes relatively larger. In contrast, in the case where the projection portions 135 are provided in correspondence with the vertices of the regular polygon RHX having 5 or more angles, the twisting of the tube 130 caused as a result of the bending force being escaped to the part that can be easily deformed is suppressed. According to this, the inclination of occlusion according to the bending direction is reduced, and occlusion of the tube 130 is prevented regardless of the bending direction.

Further, in the case where the projection portions 135 are provided in correspondence with the vertices of the regular polygon RHX having less than 5 angles and the tube 130 is bent multiple times, the tube 130 comes to have a tendency to be more easily bent in a particular direction due to the twisting of the tube 130 caused when the tube 130 is bent. Such a tendency makes the tube 130 more likely to be occluded. By providing the projection portions 135 in correspondence with the vertices of the regular polygon RHX having 5 or more angles, the twisting resulting from bending is suppressed, and thus the tube 130 is prevented from having such a tendency.

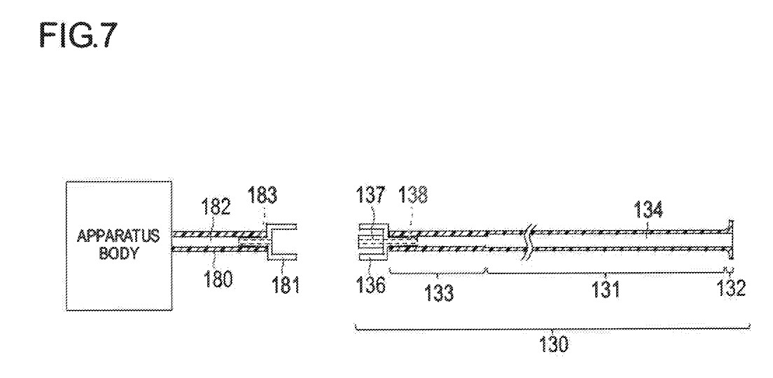

FIG. 7 illustrates a configuration of the tube 130 in which a relay portion 136 is provided on an end of the straight portion 133 of the tube 130. In FIG. 7, the apparatus body of the blood pressure measurement apparatus 100, a body-side tube 180 connected to the apparatus body, and a relay connector 181 provided on an end of the body-side tube 180 are also illustrated.

The tube 130 includes the relay portion 136 on an end of the straight portion 133. The body-side tube 180 and the tube 130 communicate with each other by connecting the relay portion 136 with the relay connector 181 provided on the end of the body-side tube 180.

The relay portion 136 and the relay connector 181 may be connected by, for example, providing the relay portion 136 and the relay connector 181 with a projection structure and a recess structure, respectively, and inserting the projection structure in the recess structure. In addition, the relay portion 136 and the relay connector 181 may respectively include a through hole 137 and a through hole 183 for the air flow path 134 of the tube 130 and an air flow path 182 of the body-side tube 180 to communicate with each other when the relay portion 136 and the relay connector 181 are connected with each other.

The configuration of the relay portion 136 and the relay connector 181 is not limited to the configuration described above. For example, the relay portion 136 may be provided with a recess structure and the relay connector 181 may be provided with a projection structure.

The shape of the inner circumference of the body-side tube 180 in a cross section is preferably formed to be circular similarly to the straight portion 133 of the tube 130.

The relay portion 136 may be provided with an insertion portion 138, and the insertion portion 138 may be inserted in the end of the straight portion 133 of the tube 130. In this way, the relay portion 136 may be provided on the end of the straight portion 133 of the tube 130.

The relay portion 136 may be integrally formed in one piece with the tube 130. In other words, the relay portion 136 may be molded at the same time with molding of the tube 130 from the same material as the tube 130.

The present embodiment achieves the following effects.

In the shape of the inner circumference in the cross section of the tube, plural projection portions projecting in correspondence with vertices of the regular polygon having 5 or more angles and inscribed in the shape of the outer circumference in the cross section are provided. According to this, when the tube is bent, twisting of the tube occurring as the result of the bending force escaping to the part that can be easily deformed is suppressed. Thus, inclination of occlusion according to the bending direction is reduced, and occlusion of the tube is prevented regardless of the bending direction.

Further, the shape of the inner circumference in the cross section of the tube is configured as the shape in which the projection portion is provided in each side of another regular polygon having angles of the same number as the regular polygon inscribed in the outer circumference. According to this, the inclination of occlusion according to the bending direction is effectively reduced, and occlusion of the tube is effectively prevented regardless of the bending direction.

Further, the projection portions are each configured to have such a shape as to project in the semicircular shape in each side of another regular polygon in the shape of the inner circumference in the cross section. According to this, a sufficient section area for air to pass through is secured regardless of the bending direction, and thus an effect of preventing occlusion of the tube resulting from bending can be enhanced.

Further, the tube does not have the projection portions in the shape of the inner circumference in the cross section in a region within the predetermined distance from one end thereof in the longitudinal direction. According to this, it is not required that a connector of the apparatus body is provided with recesses corresponding to the projection portions and the tube is connected to the connector by inserting the tube in the connector by matching the projection portions with the recesses. Thus, complication of the connection process can be prevented, and connectivity as good as a normal tube can be maintained.

Further, the flange is provided in the collar shape on the periphery of the ocher end of the long shape. According to this, it is not required to additionally produce a component including a flange and connecting the tube with the component, and thus the cost can be reduced.

Further, the plural projection portions in the shape of the inner circumference in the cross section of the tube are provided in correspondence with vertices of the regular hexagon inscribed in the outer circumference in the cross section. According to this, the area of the flow path through which air in the tube passes when the tube is bent can be increased, and thus occlusion of the tube resulting from bending the tube can be prevented more certainly.

Further, the relay portion is provided on the end of the long shape in which the projection portions are not provided in the shape of the inner circumference in the cross section. The relay portion is communicable with the body-side tube by being connected to the relay connector provided on the end of the body-side tube, and the body-side tube is connected to the body of the blood pressure measurement apparatus. According to this, the tube can be connected to the body-side tube connected to the body of the blood pressure measurement apparatus easily and without causing an air leakage.

The tube for the blood pressure cuff and the blood pressure cuff according to the embodiments of the presently disclosed subject matter has been described above. However, the presently disclosed subject matter is not limited to the embodiments.

For example, although the cuff has been described to be wrapped to the upper arm of the patient in the embodiment described above, the cuff may be wrapped to other parts than the upper arm. For example, the cuff may be wrapped to a thigh or to a lower thigh.

In the embodiment described above, the flange is formed in the collar shape on the periphery of one end of the tube, and the flange having the collar shape is fused with the periphery of the small opening portion of the bladder. However, a tube on the periphery of which the flange is not provided may be used, the flange of the collar shape may be formed by deforming one end of the tube by heat, and the formed flange may be fused with the periphery of the small opening portion of the bladder.

The apparatus body of the blood pressure measurement apparatus may be a transmitter having a transmission function for transmitting the measured value of blood pressure.

Although the regular polygon having 5 or more angles and inscribed in the outer circumference in the cross section of the tube has been described in the embodiment described above, an approximately regular polygon is also included in the scope of the presently disclosed subject matter as long as the effect of the presently disclosed subject matter is achieved.

What is claimed is:

1. A tube for a blood pressure cuff comprising:
a hollow and long shape; and
plural projection portions projecting toward a center of a regular polygon in correspondence with vertices of said regular polygon which inscribes in an outer circumference and has 5 or more angles, in a shape of an inner circumference in a cross section perpendicular to a longitudinal direction of the tube.

2. The tube for the blood pressure cuff according to claim 1, wherein
the shape of the inner circumference in the cross section comprises the projection portions in respective sides of another regular polygon having angles of the same number as said regular polygon.

3. The tube for the blood pressure cuff according to claim 2, wherein
the projection portions each project in a semicircular shape in each side of said another regular polygon in said shape of the inner circumference in the cross section.

4. The tube for the blood pressure cuff according to claim 1, wherein
said shape of the inner circumference in the cross section does not include said projection portions in a region within a predetermined distance from one end of said long shape in said longitudinal direction.

5. The tube for the blood pressure cuff according to claim 4, further comprising:
a flange provided in a collar shape on a periphery of another end of said long shape.

6. The tube for the blood pressure cuff according to claim 1, wherein
said regular polygon is a regular hexagon.

7. The tube for the blood pressure cuff according to claim 4, further comprising:
a relay portion on said one end of said long shape, the relay portion being communicable with a body-side tube by being connected to a relay connector provided on an end of said body-side tube, the body-side tube being connected to a body of a blood pressure measurement apparatus.

8. The tube for the blood pressure cuff according to claim 1, wherein
the shape of inner circumference in the cross section is circular in a region within a predetermined distance from one end of the long shape in the longitudinal direction.

9. A blood pressure cuff comprising the tube for the blood pressure cuff according to claim 1.
